(19) United States
(12) Patent Application Publication
(10) Pub. No.: US 2025/0295682 A1
(43) Pub. Date: Sep. 25, 2025

(54) USE OF NUCLEOTIDE OF LACTOBACILLUS FERMENTUM GM-090 FOR PREPARING PHARMACEUTICAL COMPOSITION FOR REGULATING IMMUNITY

(71) Applicant: GenMont Biotech Incorporation, Tainan City (TW)

(72) Inventors: Wan-Hua Tsai, Kaohsiung City (TW); Yi-Ting Fang, Tainan City (TW); Bai-Chia Liu, Tainan City (TW); Tzong-Yi Lee, Hsinchu City (TW); Jhih-Hua Jhong, Miaoli County (TW)

(21) Appl. No.: 18/791,967

(22) Filed: Aug. 1, 2024

(30) Foreign Application Priority Data
Mar. 21, 2024 (TW) 113110427

(51) Int. Cl. A61K 31/7052; A61K 35/747; A61P 29/00; A61P 37/08

(52) U.S. Cl. CPC A61K 31/7052; A61K 35/747; A61P 29/00; A61P 37/08

(57) ABSTRACT

A use of nucleotide fragments of Lactobacillus fermentum (also called Limosilactobacillus fermentum) GM-090, for preparing a composition for regulating immunity; wherein the Lactobacillus fermentum GM-090 has a deposit number of CCTCC M 204055. Moreover, the present invention further provides a composition comprising the Lactobacillus fermentum GM-090 or its nucleotide fragments thereof as an active ingredient and having an efficacy of regulating immunity.

### REFERENCE TO RELATED APPLICATIONS

The present application is based on, and claims priority from, Taiwan application number 113110427, filed Mar. 21, 2024, the disclosure of which is hereby incorporated by reference herein in its entirety.

### REFERENCE TO AN ELECTRONIC SEQUENCE LISTING

The contents of the electronic sequence listing (2024-08-01-SequenceListing.xml; Size: 18 kb; and Date of Creation: Jul. 11, 2024) is herein incorporated by reference in its entirety.

### BACKGROUND OF THE INVENTION

Bacteria have nucleic acids that can cause immune responses, called immunostimulatory sequence (ISS) oligodeoxy nucleotides (ODNs) (ISS-ODN) or immunosuppressive motifs, which carry a fragment of specific unmethylated CpG motifs that can stimulate mouse B cells to produce immune responses. Other studies have also found that ISS-ODNs without typical CpG motifs also have immunostimulatory abilities. Immunostimulatory sequence oligodeoxy nucleotides can induce innate immune responses via TLR9, including stimulating NK production, stimulating plasmacytoid DC to produce IFN-α, stimulating B cell or NK cell activation and IFN-γ production. Well-known immunostimulatory oligodeoxynucleotide (ISS-ODN) core sequences with a typical CpG motif include TTAGGG, TTTCGTTT and TCAAGCTTGA. In addition, CpG oligodeoxy nucleotides ODN2216 with its core sequence GACGATCGTC and ODN2336 with its core sequence ACGACGTCGT are known to activate human TLR-9. These two oligodeoxy nucleotides have a strong immunostimulatory ability, stimulating the production of IFN-α by B cells or dendritic cells, respectively. However, since the sequences before and after the core sequences can affect the immune efficacy of core sequence, further experiments are still needed to confirm whether different sequences have specific immunomodulatory abilities.

Therefore, the identification of oligodeoxy nucleotide fragments of probiotics with specific immunomodulatory efficacy is the subject matter to be solved by the present invention.

### FIELD OF THE INVENTION

The present invention relates to a nucleotide of Lactobacillus fermentum (also called Limosilactobacillus fermentum) GM-090 having the efficacy of regulating immunity, especially the efficacy of anti-allergic immune regulation.

### SUMMARY OF THE INVENTION

In view of this, after years of research, the inventors finally successfully isolate oligodeoxy nucleotide fragments of probiotics with the efficacy of regulating immunity. This probiotic is Lactobacillus fermentum, and its oligodeoxy nucleotide fragments have the efficacy of regulating immunity, which provide a novel and safe prevention and improvement strategy for improving allergy and inflammation.

In order to achieve the above purpose, the present invention provides a nucleotide with the effect of regulating immunity, the nucleotide sequence is set forth in SEQ ID NO: 7.

The present invention additionally provides a nucleotide with the effect of regulating immunity; the nucleotide sequence is set forth in SEQ ID NO: 13.

The present invention furthermore provides a composition with the effect of regulating immunity, which comprises Lactobacillus fermentum GM-090 or its nucleotide fragments thereof as an effective ingredient. The deposit number of the Lactobacillus fermentum GM-090 is CCTCC M 204055, and the sequence of the nucleotide fragments comprises SEQ ID NO. 7 or SEQ ID NO. 13.

In one embodiment of the present invention, the composition is in the form of a pharmaceutical composition, a nutritional supplement, or a health food.

In one embodiment of the present invention, the composition is in the form of a solution, a suspension, an emulsion, a powder, a tablet, a pill, a syrup, a lozenge, a troche, a chewing gum, a slurry, or a capsule.

In one embodiment of the present invention, the composition may further comprise an edible material, comprising water, a fluid dairy product, a milk, a concentrated milk, a yogurt, a sour milk, a frozen yogurt, a Lactobacillus fermented beverage, a milk powder, an ice cream, a butter, a cheese, a soymilk, a fermented soymilk, a fruit and vegetable juice, a fruit juice, a sports beverage, a dessert, a jelly, a candy, an infant food, a healthy food, an animal feed, a Chinese medicinal herb, or a dietary supplement.

The present invention further provides a use of nucleotide fragments of Lactobacillus fermentum GM-090 for preparing composition for regulating immunity, the composition comprising Lactobacillus fermentum GM-090 or a nucleotide fragment thereof as an active ingredient. The deposit number of the Lactobacillus fermentum GM-090 is CCTCC M 204055, and the sequence of the nucleotide fragments comprises SEQ ID NO. 7 or SEQ ID NO. 13.

In one embodiment of the present invention, the regulation of immunity involves the effect of anti-allergy and anti-inflammation.

In one embodiment of the present invention, the effects of anti-allergy and anti-inflammation are achieved by stimulating secretion of cytokines IFN-7, TL-12 and IL-10, thereby achieving the anti-allergy and anti-inflammatory effects.

This patent aims to evaluate the effect of specific oligodeoxy nucleotide fragments of Lactobacillus fermentum GM-090 on regulating immunity, and the oligodeoxy nucleotide fragments IM1: TTAGGG, IM2: TTTCGTTT, IM3: TCAAGCTTGA, ODN2216: GACGATCGTC and ODN2336: ACGACGTCGT were selected as analysis targets. Furthermore, 13 oligodeoxy nucleotide fragments with ODN2216 as the core sequence and 2 oligodeoxy nucleotide fragments with ODN2336 as the core sequence were further tested experimentally for their efficacies of regulating immunity. The results show that GM-090 with 2 specific oligodeoxy nucleotide fragments has the efficacy of regulating immunity. The composition provided by the present invention has the advantage of low side effects due to the use of probiotics or nucleotides as an active ingredient, which provides a novel and safe prevention and improvement strategy for anti-allergy, regulation of immunity and anti-inflammation.

### BRIEF DESCRIPTION OF THE DRAWINGS

The techniques of present invention would be more understandable from the detailed description given herein below and the accompanying figures are provided for better illustration, and thus description and figures are not limitative for present invention, and wherein:

FIG. 1A shows the full gene profile of the assembled GM-090.

FIG. 1B shows the genetic information of GM-090.

FIG. 1C shows the results and genome features of GM-090 by whole genome sequencing.

FIG. 1D shows the results and genome features of GM-090 by whole genome sequencing.

FIG. 2A shows comparisons of the abilities of synthetic sequences of ISS-ODNs of GM-090 to stimulate production of IFN-7.

FIG. 2B shows comparisons of the abilities of synthetic sequences of ISS-ODNs of GM-090 to stimulate production of IFN-7.

FIG. 3A shows comparisons of the abilities of synthetic sequences of ISS-ODNs of GM-090 to stimulate production of IL-12.

FIG. 3B shows comparisons of the abilities of synthetic sequences of ISS-ODNs of GM-090 to stimulate production of IL-12

FIG. 4A shows comparisons of the abilities of synthetic sequences of ISS-ODNs of GM-090 to stimulate production of IL-10.

FIG. 4B shows comparisons of the abilities of synthetic sequences of ISS-ODNs of GM-090 to stimulate production of IL-10.

### DETAILED DESCRIPTION OF THE INVENTION

All technical and scientific terms used in this specification have the meaning commonly understood by those skilled in the art to which the present disclosure belongs, unless otherwise defined.

The singular phrases “a”, “an”, and “the” used in the specification and claims can refer to more than one object, unless otherwise stated.

The phrases “or”, “as well as”, and “and” used in this specification all refer to “or/and”, unless otherwise stated. In addition, the phrases “comprise” and “include” are not open-ended conjunctions with restrictions. The foregoing paragraphs are only for systematic purposes and should not be interpreted as limitations on the subject matter of the present invention.

The composition provided by the present invention can be prepared into a dosage form suitable for the composition of the invention by combining an active ingredient or a composition provided by the invention with at least one pharmaceutically acceptable vehicle using techniques well known to those skilled in the art to which the invention belongs. The dosage form includes, but is not limited to, solution, emulsion, suspension, powder, tablet, lozenge, pill, chewing gum, capsule, and other similar or suitable dosage forms for the present invention.

The term “pharmaceutically acceptable” means that the substance or composition must be compatible with the other ingredients of its pharmaceutical formulation, and does not exacerbate the symptoms of the patient.

The term “pharmaceutically acceptable vehicle” includes one or more ingredient types selected from solvents, emulsifiers, suspending agents, disintegrants, binders, excipients, stabilizers, chelating agents, diluents, gelling agents, preservatives, lubricants, surfactants and other similar or suitable vehicles for the present invention.

In the aforementioned composition, one or more dissolution aids, buffers, colorants, flavoring agents and the like, which are generally used in the field of preparations, may also be appropriately added as needed.

The term “pharmaceutical composition” refers to a solid or liquid composition in form, concentration and degree of purity suitable for administration to patients, which can induce desired physiological changes after administration. The pharmaceutical composition is sterile and/or non-pyrogenic.

Unless otherwise specified, the materials used in the present invention are all commercially available materials. The strain GM-090 of Lactobacillus fermentum (also called Limosilactobacillus fermentum), used in embodiments of the present invention is deposited at the Bioresource Collection and Research Center of Food Industry Research and Development Institute in Taiwan under deposit number BCRC 910259 and China Center for Type Culture Collection (CCTCC) under deposit number CCTCC M 204055.

### Example 1. Whole Genome Sequencing of GM-090

Culture and sequencing of probiotic strain: 1 ml of lactic acid bacteria cultured overnight were taken for second-generation culture using 0.1 ml to 10 ml MRS broth while observing growth curve. After the bacteria strain grew to OD600 of 0.8, 3 ml bacteria solution was taken and washed twice with filtered sterile water (13000 rpm, 1 min). Finally, bacterial cells were left for subsequent genomic DNA extraction using QIAGEN DNeasy® Blood & Tissue Kit (QIAGEN; Cat. No. 69504). After the quality of genomic DNA was confirmed by using Qubit fluorometer, nanophotometer and agarose gel, the Whole-genome DNA sequencing was subsequently performed by Town Health Company. The resulting DNAs to be tested were simultaneously sequenced using Illumina Hiseq 2000 next-generation sequencer and Oxford Nanopore GridION third-generation sequencer.

Treatment before sequence assembly: Before sequence assembly, FASTX-Toolkit and MinIONQC tools were used to filter low-quality noise from the sequencing data, and Quality Value=20, that is, the per-base error rate was 1/100.

Sequence assembly and correction of sequencing data: The nucleotide sequences of the filtered high-quality next-generation sequencing short fragment were combined with the sequences of the third-generation sequencing data to form longer continuous sequences by hybrid assembly using published tool MaSuRCA v3.3.1, and the sequences of the third-generation sequencing data were used to sequence alignment to construct the bridging sequences and confirm a location relationship between contigs. Furthermore, Pilon was used to complete variation detection and improvement of genome assembly. Based on the Nanopore long-read sequence as a reference sequence, after short-read alignment, the sequences were detected and corrected for single-base differences and small gaps for each reading to reduce false positives.

Species evolution analysis, sequenced gene annotation and functional pathway analysis: The assembled sequences were sequenced, and multiple sequence alignment of the collected sequences of subtype strains and the sequences to be identified was performed, thereby completing the important basis of molecular evolution analysis, phylogenetic analysis and traceability, in order to achieve the purpose of rapid and accurate identification of bacterial strains using next-generation sequencing platforms. The genome was annotated using a number of tools, and Prokka was used to predict genes in the whole genome of prokaryotes, including protein coding region and non-coding region. Plasflow was used for plasmid identification. A PHASTER tool was used to screen phage segments in genome. Bagel4 and CARD predicted genes associated with the production of bacteriocins and possible regions of drug resistance, respectively. The protein coding region is a segment that may be translated into a functional protein. Functional classification is annotated and classified by an eggNOG tool in combination with the Cluster of Orthologous Genes (COG) of protein databank.

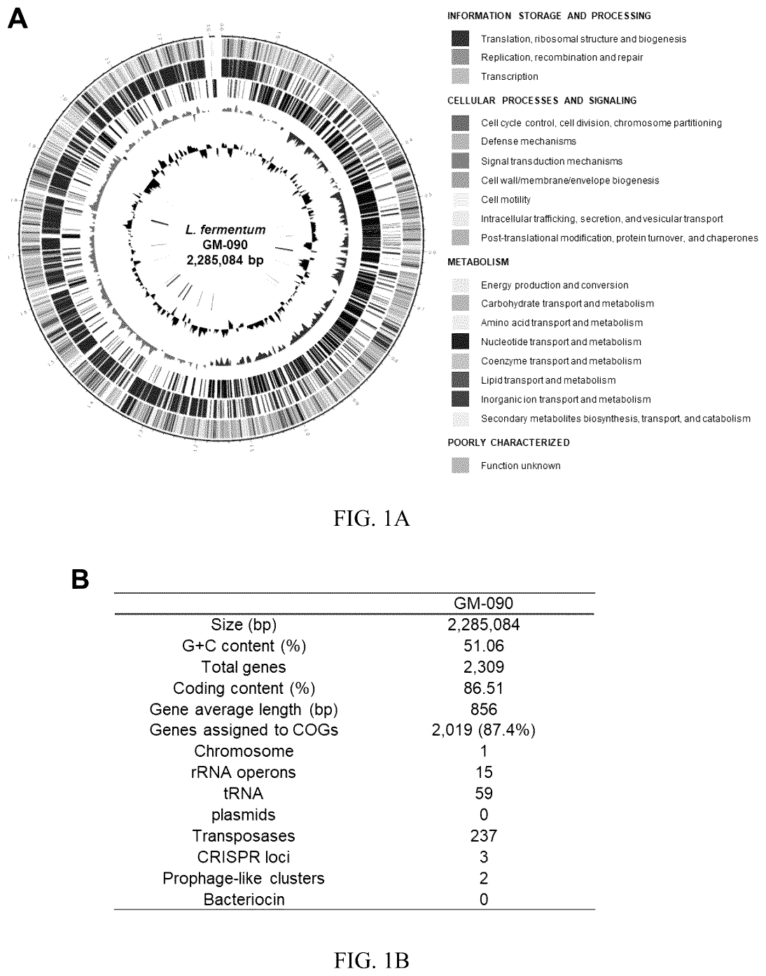

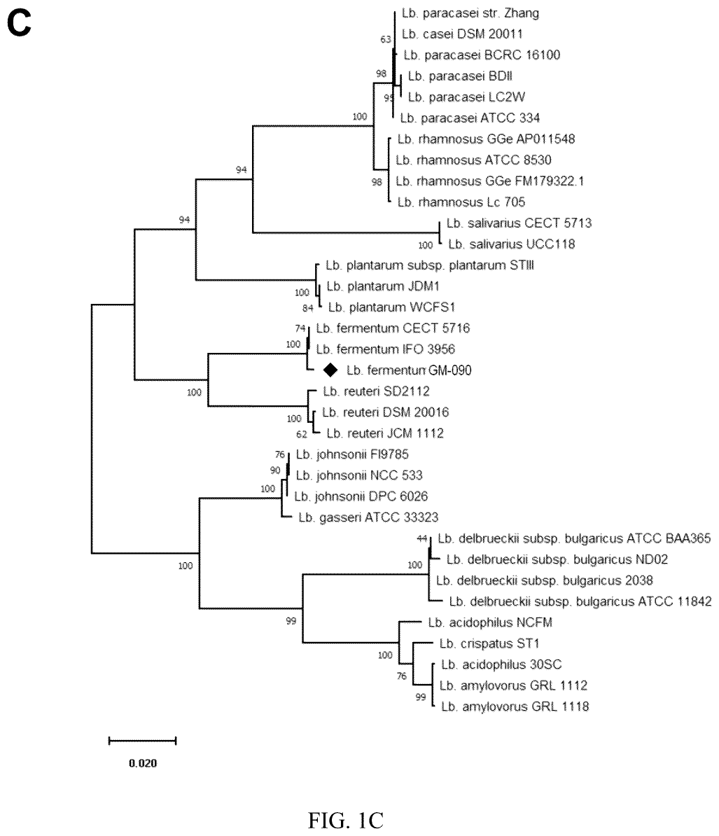

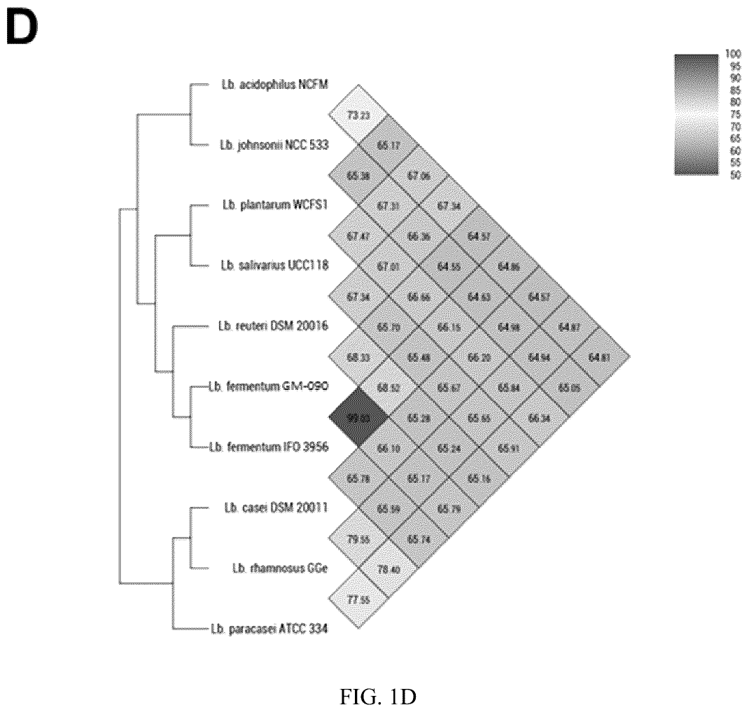

An Illumina Hiseq 2000 next-generation sequencer and an Oxford Nanopore GridION third-generation sequencer were used for gene sequencing of GM-090, followed by whole genome assembly and subsequent functional analysis. FIG. 1A shows the full gene profile of the assembled GM-090, with a size of 2,285,084 bp. The genetic information of GM-090 is shown in FIG. 1B. The strain GM-090 was determined to be Lactobacillus fermentum, which is extremely similar to other Lactobacillus fermentum with published whole genome sequences (FIGS. 1C and D). Previous studies have found that probiotics containing specific immunostimulatory sequence oligodeoxy nucleosides (ISS-ODNs) from their whole gonome have the effect to stimulate the host immune response. We compared the sequences of ISS-ODNs in GM-020 with those in other strains of Lactobacillus fermentum, including FTDC 8312, CBA7106 and LDTM 7301, using whole genome sequencing. The compared sequences of ISS-ODNs included IM1 (TTAGGG), IM2 (TTTCGTTT), IM3 (TCAAGCTTGA), ODN2216 (GACGATCGTC), and ODN2336 (ACGACGTCGT). The occurrence frequencies of these ISS-ODNs in the whole genome of GM-090 and the other three strains of Lactobacillus fermentum are shown in Table 1. IM1(TTAGGG) and IM2 (TTTCGTTT) appeared a higher frequencies in GM-090 than in other strains, indicating that GM-090 has a relatively stronger immunomodulatory ability.

TABLE 1
Comparison of the occurrence frequency of ISS-ODNs in different
Lactobacillus fermentum strains

| | | | | FTDC | | LDTM |
|---|---|---|---|---|---|---|
| | | | GM-090 | 8312 | CBA7106 | 7301 |
| Code | Sequence | Genome | 2,285,084 | 2,239,920 | 2,042,280 | 2,046,200 |
| | | size (bp) | | | | |
| IM1 | TTAGGG | Frequency | 546 | 451 | 464 | 470 |
| | | No. copies | 23.9 | 20.1 | 22.7 | 23 |
| | | per 106 | | | | |
| | | bases | | | | |
| IM2 | TTTCGTTT | Frequency | 56 | 46 | 47 | 47 |
| | | No. copies | 2.45 | 2.05 | | 2.3 |
| | | per 106 | | | | |
| | | bases | | | | |
| IM3 | TCAAGCTTGA | Frequency | 1 | 3 | 2 | 1 |
| | | No. copies | 0.04 | 0.13 | 0.1 | 0.05 |
| | | per 106 | | | | |
| | | bases | | | | |
| ODN2216 | GACGATCGTC | Frequency | 13 | NA | NA | NA |
| | | No. copies | 0.57 | NA | NA | NA |
| | | per 106 | | | | |
| | | bases | | | | |
| ODN2336 | ACGACGTCGT | Frequency | 2 | NA | NA | NA |
| | | No. copies | 0.09 | NA | NA | NA |
| | | per 106 | | | | |
| | | bases | | | | |

NA: no analysis

GM-090 also appeared 13 frequencies of ODN2216 (GACGATCGTC) and 2 frequencies of ODN2336 (ACGACGTCGT) in different locations of whole genome. Although the core sequence of ODN2336 (GACGATCGTC) are the same across the 13 genome locations, the 4-6 nucleotides at front and rear ends of the core sequence are completely different. The variations were also observed in core sequence of ODN2336 (ACGACGTCGT) at 2 genome locations, as shown in Table 2. Based on the previous analysis of whole-genome sequencing in Lactobacillus paracasei GM-080 and Lactobacillus rhamnosus GM-020, GM-080 and GM-020 showed only 1 to 2 occurrence of ODN 2216 core sequences. In contrast, GM-090 containing 13 occurrence of ODN221 could highly correlated with its immunomodulatory ability.

TABLE 2
Synthetic sequences of ISS-ODNs of GM-090

| | core sequence | sequence | Sequence ID | in vitro |
|---|---|---|---|---|
| IM1 | TTAGGG | | | NA |
| IM2 | TTTCGTTT | | | NA |
| IM3-1 | TCAAGCTTGA | CGCGAATCAAGCTTGATCTT | SEQ ID NO: 1 | yes |
| ODN-1 | GACGATCGTC | TAATGACGATCGTCGGGATT | SEQ ID NO: 2 | yes |
| ODN-2 | | CGTTGACGATCGTCGTTTGG | SEQ ID NO: 3 | yes |
| ODN-3 | | CGCTGACGATCGTCAACAGG | SEQ ID NO: 4 | yes |
| ODN-4 | | ACCCGACGATCGTCAAGGAG | SEQ ID NO: 5 | yes |
| ODN-5 | | CCGGGACGATCGTCGCTGAC | SEQ ID NO: 6 | yes |
| ODN-6 | | GCTTGACGATCGTCTTGATC | SEQ ID NO: 7 | yes |
| ODN-7 | | AGACGACGATCGTCAGCATT | SEQ ID NO: 8 | yes |
| ODN-8 | | ACGTGACGATCGTCTCAGAG | SEQ ID NO: 9 | yes |
| ODN-9 | | CGCTGACGATCGTCGGTAGC | SEQ ID NO: | yes |
| ODN- | | TTAAGACGATCGTCGGCAAG | SEQ ID NO: | yes |
| 10 | | | 11 | |
| ODN- | | ACCTGACGATCGTCTTTGGC | SEQ ID NO: | yes |
| 11 | | | 12 | |
| ODN- | | ACGCGACGATCGTCGAACGG | SEQ ID NO: | yes |
| 12 | | | 13 | |
| ODN- | | TCCTGACGATCGTCGATAAC | SEQ ID NO: | yes |
| 13 | | | 14 | |
| ODN- | ACGACGTCGT | TCCAACGACGTCGTGGGCATT | SEQ ID NO: | yes |
| 14 | | | 15 | |
| ODN- | | GCAAACGACGTCGTTCCCTTT | SEQ ID NO: | yes |
| 15 | | | 16 | |

NA: no analysis

### Example 2. Pretreatment of ISS-ODNs

The total of 16 sequences of IM3-1 and ODN-1 to ODN-15 were synthesized by MDBio, Inc., Taiwan. The sequences are shown in Table 2 above. The DNA powder received was rapidly centrifuged, and diluted to 200 μM with physiological saline for injection according to the volume provided by the synthesizer company. After standing for 10 min, the solution was serially diluted in half-dilution to obtain half-diluted stock liquids with concentrations of 100 μM to 6.25 μM.

### Example 3. Analysis of Immunity Regulation Using Mouse Splenocytes

Previous studies have shown that IFN-γ and IL-10 have negative feedback response activity in Th2 cells and can down-regulate specific IgE production. IL-12 can trigger the Th2 response into a dominant Th1 response, which is potentially useful for treatment. IFN-γ, IL-12, and IL-10 are considered as anti-allergy-related cytokines.

After the mice were sacrificed, spleens were placed in 10 ml culture medium and ground into suspension with frosted glass slide. The suspension was filtered with a 70 m strainer, and centrifuged at 400 g for 5 min at 4° C. to remove the culture medium. The cell clumps were broken up and added 3 ml ACK lysis buffer, and then left at 37° C. for 3 min. After that, 7 ml culture medium was added and mixed, and centrifuged at 400 g for 5 min at 4° C. to remove the culture medium. 10 ml fresh culture medium was added, mixed, and washed twice. The cell suspension was removed and stained with Trypan blue stain to calculate cell concentration. The cell concentration was adjusted to 4×10⁶ cells/ml, and then added 100 μl of cell suspension to each well in a 96-well plate. Then 100 μl culture medium was added to a well as a control group (0 PM). The substances to be tested were adjusted to appropriate concentrations and added 100 μl to the wells. The 96-well plate was placed in a cell incubator at 37° C. with CO₂ for 48 h. The final concentrations of ISS-ODNs were 0.125, 0.25, 0.5, 1, and 2 M. The 96-well plate was centrifuged at 3000 rpm for 10 min, and then supernatants were added into 1.5 mL centrifuge tubes for subsequent ELISA kit analysis, including IFN-γ (BD OptEIATM, Mouse IFN-γ ELISA Set, Cat: 555138), IL-12 (BD OptEIATM, Mouse IL-12 (p70) ELISA Set, Cat: 555256), and IL-10 (BD OptEIATM, Mouse IL-10 ELISA Set, Cat: 555252).

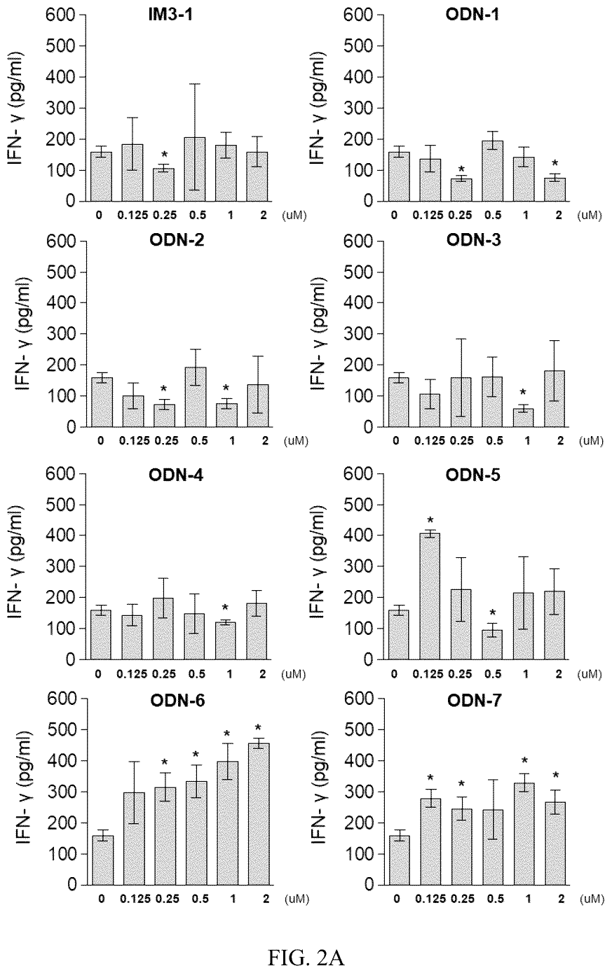

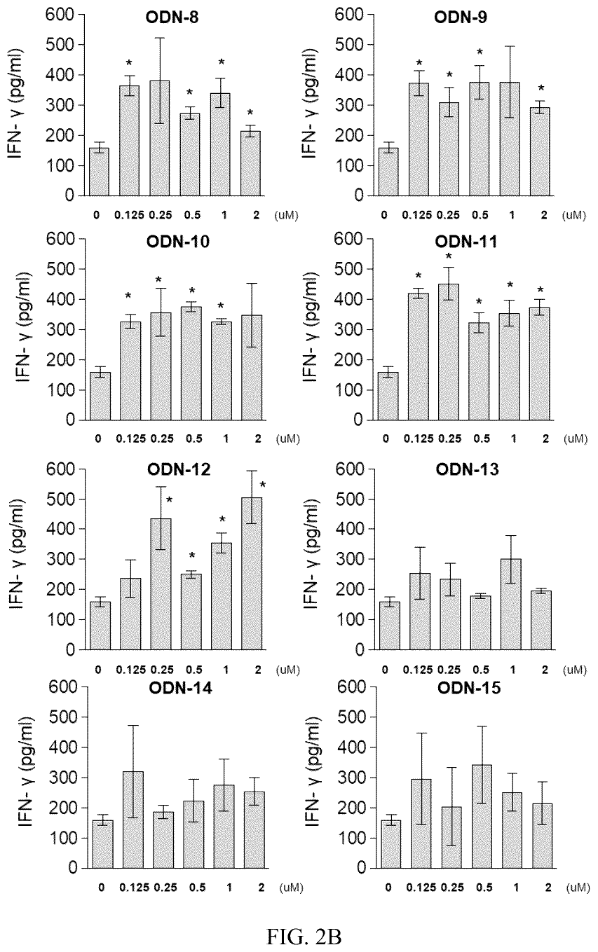

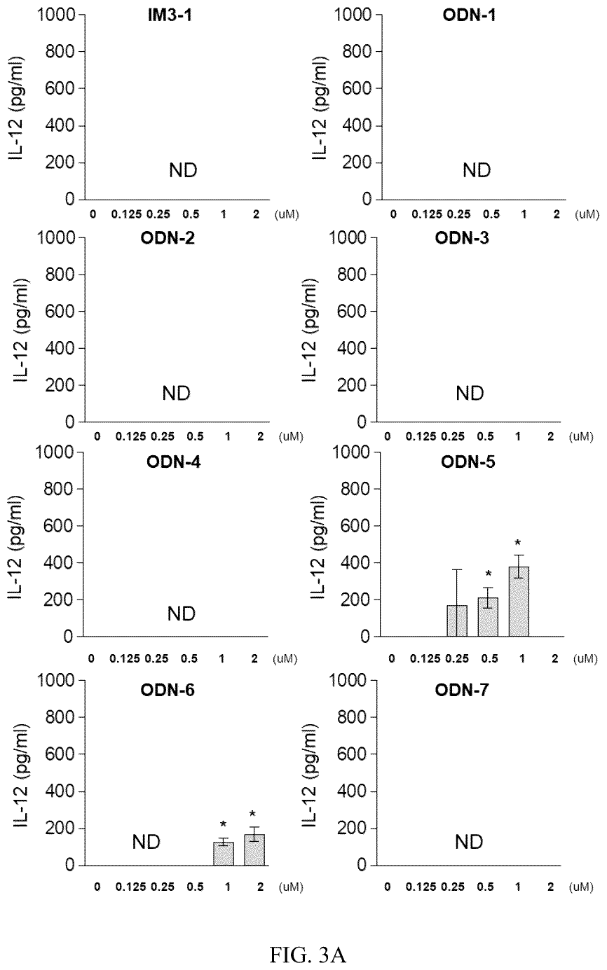

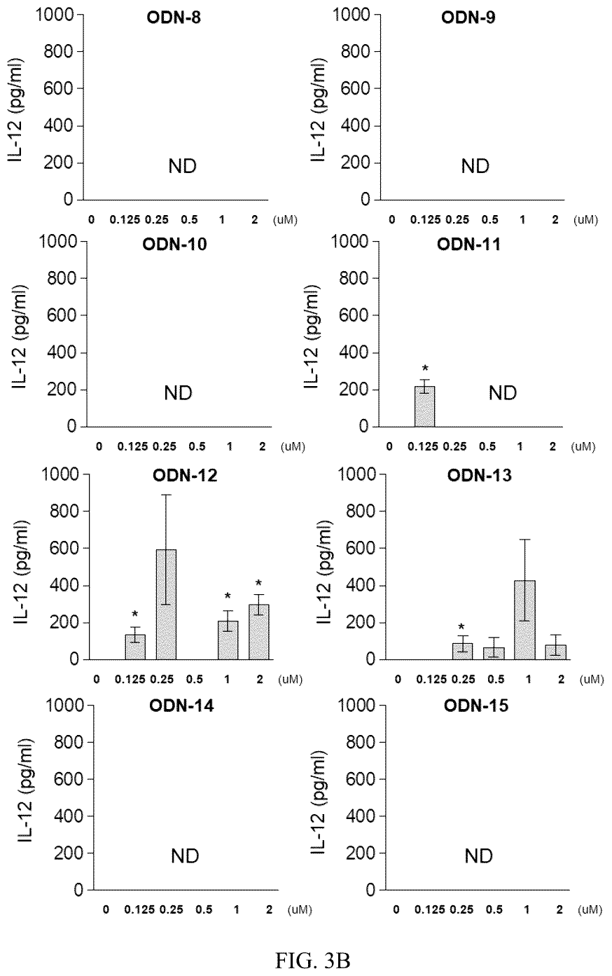

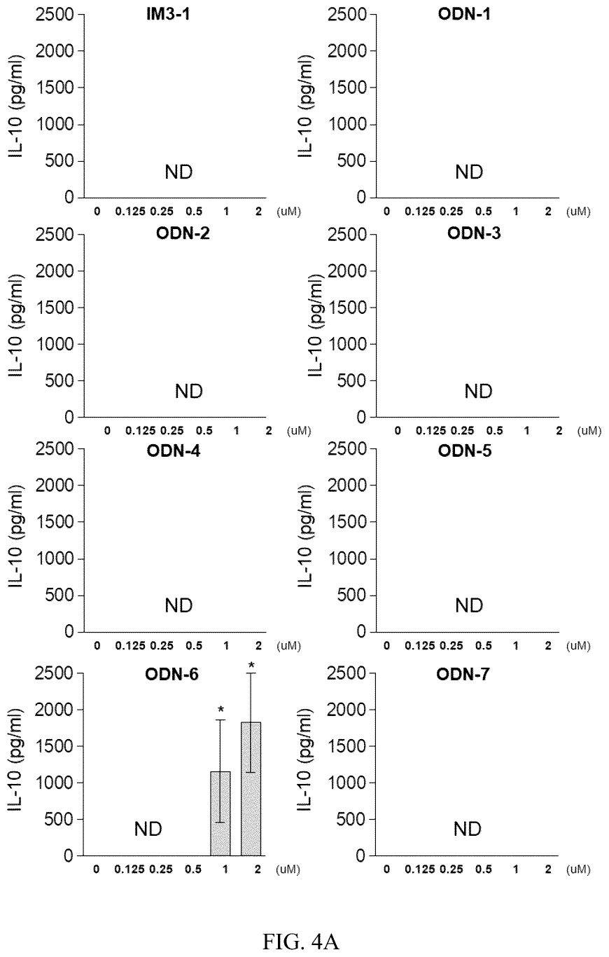

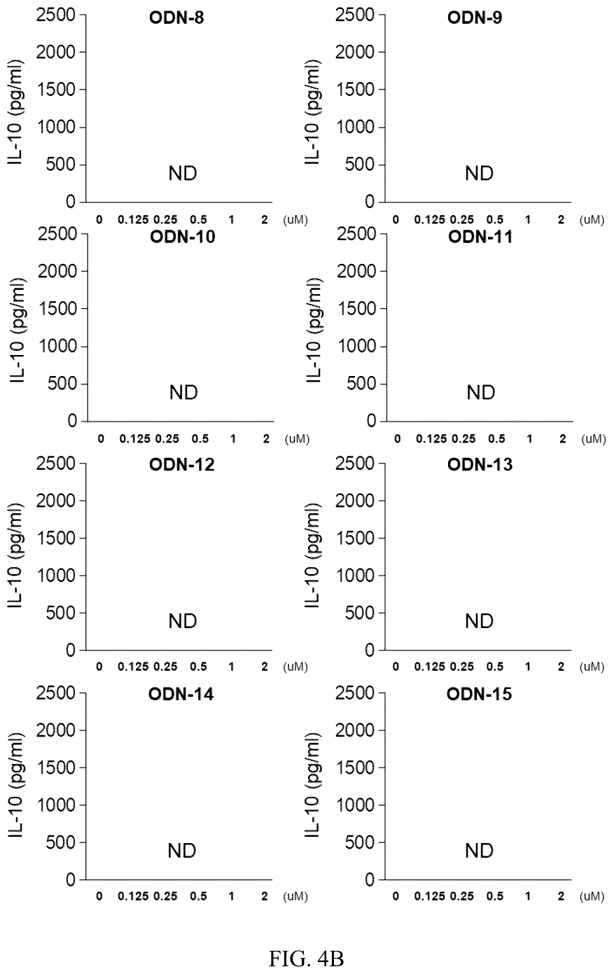

The ability of specific ISS-ODNs (IM3-1 and ODN-1 to ODN-15) in GM-090 to stimulate the secretion of anti-allergy-related cytokines IFN-γ, IL-12, and IL-10 was further confirmed by using mouse splenocytes. Therefore, after splenocytes were treated with the synthetic nucleotides at different concentrations, the secretion of these cytokines was observed. The results show that ODN-6, -7, -8, -9, -10, -11 and -12 of GM-090 could increasingly stimulate the production of IFN-γ in splenocytes (FIG. 2), with the highest concentration of IFN-γ being stimulated by ODN-6, -11 and -12. While ODN-5, -6, -11 and -12 of GM-090 also showed the ability to stimulate IL-12 production in splenocytes (FIG. 3), the stimulation ability showed no apparent difference among the groups of ODN-5, -6, -11, and -12. Specifically, among these 16 ODNs of GM-090, only ODN-6 could stimulate the production of anti-inflammatory cytokine IL-10 in splenocyte at the dosage of 1-2 M (FIG. 4). The results are shown and summarized in Table 3, indicating that GM-090 contained specific ISS-ODNs such as ODN-6, which stimulated the production of IFN-γ, IL-12 and IL-10; and ODN-12, which stimulated the production of IFN-γ and IL-12. The cytokines play the crucial role in regulating host immunity, contributing to effects of anti-allergy, immunity regulation and anti-inflammation.

TABLE 3
The effective dosages of ISS-ODNs
of GM-090 in immunol-modulation

| μM | IFN-γ | IL-12 | IL-10 |
|---|---|---|---|
| IM3-1 | ND | ND | ND |
| ODN-1 | ND | ND | ND |
| ODN-2 | ND | ND | ND |
| ODN-3 | ND | ND | ND |
| ODN-4 | ND | ND | ND |
| ODN-5 | ND | 0.5-1 | ND |
| ODN-6 | 0.25-2 | 1-2 | 1-2 |
| ODN-7 | 0.125-2 | ND | ND |
| ODN-8 | 0.125-2 | ND | ND |
| ODN-9 | 0.125-2 | ND | ND |
| ODN-10 | 0.125-1 | ND | ND |
| ODN-11 | 0.125-2 | 0.125 | ND |
| ODN-12 | 0.25-2 | 1-2 | ND |
| ODN-13 | ND | ND | ND |
| ODN-14 | ND | ND | ND |
| ODN-15 | ND | ND | ND |

ND: not detected

From the content disclosed in the preferred embodiments of this specification, it will be apparent to those skilled in the art to which the invention belongs that the foregoing embodiments are exemplary only. Those skilled in the art to which the invention belongs can implement the invention through many alterations and substitutions without any difference from the technical features of the present invention. According to embodiments described in this specification, various modifications may be made to the invention without hindering the implementation. The claims provided in this specification define the scope of the present invention, which covers the foregoing methods and structures as well as inventions equivalent thereto.

The above-mentioned multiple efficacies in fact fully satisfy the patentability requirements for novelty and inventive step. Therefore, this application is filed in accordance with the Patent Act. We strongly urge your office to approve this patent application for invention to encourage invention.

### Biological Material Deposit

The strain GM-090 of Lactobacillus fermentum is deposited at the Bioresource Collection and Research Center of Food Industry Research and Development Institute in Taiwan under deposit number BCRC 910259 and China Center for Type Culture Collection (CCTCC) under deposit number CCTCC M 204055.

What is claimed is:

1. A nucleotide with an effect of regulating immunity, the nucleotide sequence is set forth in SEQ ID NO: 7 or SEQ ID NO: 13.

2. A composition with an effect of regulating immunity, the composition comprising Lactobacillus fermentum GM-090 or its nucleotide fragments thereof as an effective ingredient; the Lactobacillus fermentum GM-090 has a deposit number of CCTCC M 204055, and the sequence of the nucleotide fragments comprise SEQ ID NO. 7 or SEQ ID NO. 13.

3. The composition as claimed in claim 2, wherein the composition is a pharmaceutical composition, a nutritional supplement, or a health food.

4. The composition as claimed in claim 2, wherein the composition may further comprise a pharmaceutically acceptable vehicle.

5. The composition as claimed in claim 2, wherein the composition is a solution, a suspension, an emulsion, a powder, a tablet, a pill, a syrup, a lozenge, a troche, a chewing gum, a slurry, or a capsule.

6. The composition as claimed in claim 2, wherein the composition may further comprise an edible material comprising water, a fluid dairy product, a milk, a concentrated milk, a yogurt, a sour milk, a frozen yogurt, a Lactobacillus fermented beverage, a milk powder, an ice cream, a butter, a soymilk, a fermented soymilk, a fruit and vegetable juice, a fruit juice, a sports beverage, a dessert, a jelly, a candy, an infant food, a healthy food, an animal feed, a Chinese medicinal herb, or a dietary supplement.

7. A method of nucleotide fragments or Lactobacillus fermentum GM-090 for preparing a composition for regulating immunity, the Lactobacillus fermentum GM-090 having a deposit number of CCTCC M 204055, and the nucleotide fragments contain the sequence of SEQ ID NO. 7 or SEQ ID NO. 13.

8. The method of claim 7, wherein the regulation of immunity is effects of anti-allergy and anti-inflammation.

9. The method as claimed in claim 8, wherein the effects of anti-allergy and anti-inflammation are achieved by stimulating secretion of cytokines IFN-γ, TL-12 and IL-10, thereby achieving the anti-allergic and anti-inflammatory effects.


SEQUENCE LISTING (16 sequences)

SEQ ID NO: 1 - DNA, 20 bp, Lactobacillus fermentum
  source 1..20: mol_type = genomic DNA; strain = GM-090
cgcgaatcaagcttgatctt

SEQ ID NO: 2 - DNA, 20 bp, Lactobacillus fermentum
  source 1..20: mol_type = genomic DNA; strain = GM-090
taatgacgatcgtcgggatt

SEQ ID NO: 3 - DNA, 20 bp, Lactobacillus fermentum
  source 1..20: mol_type = genomic DNA; strain = GM-090
cgttgacgatcgtcgtttgg

SEQ ID NO: 4 - DNA, 20 bp, Lactobacillus fermentum
  source 1..20: mol_type = genomic DNA; strain = GM-090
cgctgacgatcgtcaacagg

SEQ ID NO: 5 - DNA, 20 bp, Lactobacillus fermentum
  source 1..20: mol_type = genomic DNA; strain = GM-090
acccgacgatcgtcaaggag

SEQ ID NO: 6 - DNA, 20 bp, Lactobacillus fermentum
  source 1..20: mol_type = genomic DNA; strain = GM-090
ccgggacgatcgtcgctgac

SEQ ID NO: 7 - DNA, 20 bp, Lactobacillus fermentum
  source 1..20: mol_type = genomic DNA; strain = GM-090
gcttgacgatcgtcttgatc

SEQ ID NO: 8 - DNA, 20 bp, Lactobacillus fermentum
  source 1..20: mol_type = genomic DNA; strain = GM-090
agacgacgatcgtcagcatt

SEQ ID NO: 9 - DNA, 20 bp, Lactobacillus fermentum
  source 1..20: mol_type = genomic DNA; strain = GM-090
acgtgacgatcgtctcagag

SEQ ID NO: 10 - DNA, 20 bp, Lactobacillus fermentum
  source 1..20: mol_type = genomic DNA; strain = GM-090
cgctgacgatcgtcggtagc

SEQ ID NO: 11 - DNA, 20 bp, Lactobacillus fermentum
  source 1..20: mol_type = genomic DNA; strain = GM-090
ttaagacgatcgtcggcaag

SEQ ID NO: 12 - DNA, 20 bp, Lactobacillus fermentum
  source 1..20: mol_type = genomic DNA; strain = GM-090
acctgacgatcgtctttggc

SEQ ID NO: 13 - DNA, 20 bp, Lactobacillus fermentum
  source 1..20: mol_type = genomic DNA; strain = GM-090
acgcgacgatcgtcgaacgg

SEQ ID NO: 14 - DNA, 20 bp, Lactobacillus fermentum
  source 1..20: mol_type = genomic DNA; strain = GM-090
tcctgacgatcgtcgataac

SEQ ID NO: 15 - DNA, 21 bp, Lactobacillus fermentum
  source 1..21: mol_type = genomic DNA; strain = GM-090
tccaacgacgtcgtgggcatt

SEQ ID NO: 16 - DNA, 21 bp, Lactobacillus fermentum
  source 1..21: mol_type = genomic DNA; strain = GM-090
gcaaacgacgtcgttcccttt